SEQUENCE LISTING (2 sequences)

SEQ ID NO: 1 - DNA, 20 bp, synthetic construct
  source 1..20: mol_type = other DNA
agagtttgatcmtggctcag

SEQ ID NO: 2 - DNA, 20 bp, synthetic construct
  source 1..20: mol_type = other DNA
ggttacctttgttacgactt

(19) United States
(12) Patent Application Publication
(10) Pub. No.: US 2025/0170193 A1
(43) Pub. Date: May 29, 2025

(54) LACTOBACILLUS PARACASEI GLU70 STRAIN WITH GLUTEN-DEGRADING ABILITY AND ANTIBACTERIAL ACTIVITY AND PROBIOTIC PROPERTIES AND USES THEREOF

(71) Applicant: Microbiome Inc., Seoul (KR)

(72) Inventors: Bok Kun YOON, Seoul (KR); Seul Gi YOON, Seoul (KR); Jun Ho JANG, Seoul (KR); Ju Sung YOON, Seoul (KR); Eun Shim SON, Gunpo-Si, Gyeonggi-Do (KR)

(73) Assignee: Microbiome Inc., Seoul (KR)

(21) Appl. No.: 18/930,459

(22) Filed: Oct. 29, 2024

Related U.S. Application Data
(63) Continuation of application No. 18/416,083, filed on Jan. 18, 2024, now abandoned.

(30) Foreign Application Priority Data
Nov. 24, 2023 (KR) 10-2023-0165387

(51) Int. Cl. A61K 35/747; A61P 31/04

(52) U.S. Cl. CPC A61K 35/747; A61P 31/04

(57) ABSTRACT

The present invention relates to the use of a Lactobacillus paracasei GLU70 strain. Since the strain of the present invention has probiotic properties and antibacterial activity, it can be used as a probiotic composition, an antibacterial composition, and a starter composition for manufacturing fermented food.

### CROSS-REFERENCE TO RELATED APPLICATION

This application is a Continuation Application of U.S. patent application Ser. No. 18/416,083, filed on Jan. 18, 2024, which claims priority to and the benefit of Korean Patent Application No. 10-2023-0165387, filed on Nov. 24, 2023, the disclosure of which is incorporated herein by reference in its entirety.

### REFERENCE TO ELECTRONIC SEQUENCE LISTING

The contents of the Sequence-Listing XML (Sequence listing.xml; Date of Creation: Oct. 29, 2024; and Size: 2,937 bytes) is herein incorporated by reference in its entirety.

### BACKGROUND

### 1. Field of the Invention

The present invention relates to use of a Lactobacillus paracasei GLU70 strain. Since the strain of the present invention has probiotic properties and antibacterial activity, it can be used as a probiotic composition, an antibacterial composition, and a starter composition for manufacturing fermented food.

### 2. Discussion of Related Art

The word “probiotics” came from a Greek word meaning “for life.” The meaning of this word has gone through several revisions, and it is now defined as “living bacteria in the form of a single or complex strain that is fed to humans or animals in the form of dried cells or fermented products, providing beneficial effects by improving the intestinal flora of human or animal hosts.”

The probiotics are currently used in three senses in Korea: when used as a feed additive for animals, they are labeled as a live bacteria preparation, and when used as human medicine, they are labeled as a bowel preparation or lactic acid bacteria preparation.

Therefore, probiotics are characterized by live microorganisms that are beneficial to the health of the host when consumed in an appropriate amount. It has been reported that probiotics such as lactic acid bacteria (LAB) and Bifidobacteria are effective in improving intestinal health, strengthening immunity, reducing serum cholesterol, providing anti-cancer effects, and preventing coronary artery disease. As such probiotics, aerobic bacteria, anaerobic bacteria, lactic acid bacteria, and yeasts are used, but lactic acid bacteria are mainly used as the main strain.

Lactic acid bacteria are widely present in nature and produce lactic acid by using carbohydrates anaerobically. Natural environments in which lactic acid bacteria are found are diverse; they are present not only in the intestines of humans and animals, but also in various vegetables and fruits. They play an important role in the fermentation process in fermented food such as Korean kimchi, yogurt, and German sauerkraut.

After entering the intestines, these lactic acid bacteria colonize the intestinal epithelial cells, and by preventing harmful microorganisms from settling in the intestines and by secreting antibacterial substances, they not only suppress the growth of harmful microorganisms and ameliorate diarrhea and constipation, but also perform functions such as immune activity enhancement, anti-cancer actions, and serum cholesterol reduction.

In addition, it is generally known that these lactic acid bacteria are directly or indirectly added to food to improve the storability of food and the flavor and texture of food by lactic acid, a metabolite thereof. It has been reported that the main lactic acid bacteria are Lactobacillus acidophilus, Lactobacillus casei, Lactobacillus fermentum, and Lactobacillus plantarum, and these lactic acid bacteria are widely used worldwide as a starter and a probiotic preparation of fermented dairy products or the like.

Meanwhile, Korean Patent Publication No. 2023-0085279 discloses “Immunity Enhancing Composition including Composition for Preventing or Treating Cognitive Disorder and Intestinal Disorder Comprising Lactobacillus paracasei NK112, Cuscuta Australis R.Br. Extract and Cuscuta Japonica Choisy Extract”; Korean Registered Patent No. 354,315 discloses “Novel Lactobacillus sp. Producing Antibacterial Substance”; and Korean Registered Patent No. 2,587,892 discloses “Lactobacillus paracasei HY7017 strain with Enhanced Functional Characteristics and Enhanced Immunity Function by Using Red Ginseng as a Nutrient Source, and Use Thereof” However, “Lactobacillus paracasei GLU70 strain with antibacterial activity and probiotic properties and uses thereof” as in the present invention has never been disclosed.

### RELATED ART DOCUMENTS

### Patent Documents

(Patent Document 001) Korean Patent Publication No. 10-2023-0085279
(Patent Document 002) Korean Registered Patent No. 354,315
(Patent Document 003) Korean Registered Patent No. 2,587,892

### SUMMARY

The present invention relates to the use of a Lactobacillus paracasei GLU70 strain isolated from salted seafood and having antibacterial activity, acid resistance, bile resistance, heat resistance, and gluten-degrading ability, and is directed to presenting the potential of industrial applicability thereof and utilizing the same.

Therefore, the present invention is directed to providing a probiotic composition including a Lactobacillus paracasei GLU70 strain with Accession Number KCTC13415BP, a culture solution thereof, a concentrate of the culture solution, or a dried product thereof as an active ingredient.

The present invention is also directed to providing a food composition including the Lactobacillus paracasei GLU70 strain with Accession Number KCTC13415BP, a culture solution thereof, a concentrate of the culture solution, or a dried product thereof as an active ingredient.

The present invention is also directed to providing an antibacterial composition against pathogenic microorganisms, including the Lactobacillus paracasei GLU70 strain with Accession Number KCTC13415BP, a culture solution thereof, a concentrate of the culture solution, or a dried product thereof as an active ingredient.

The present invention is also directed to providing a starter composition for manufacturing fermented food, including the Lactobacillus paracasei GLU70 strain with Accession Number KCTC13415BP, a culture solution thereof, a concentrate of the culture solution, or a dried product thereof as an active ingredient.

Accordingly, the present inventors confirmed the useful biological properties of the Lactobacillus paracasei GLU70 strain, such as gluten-degrading ability, acid resistance, bile resistance, heat resistance, and antibacterial activity, and completed the present invention.

For the Lactobacillus paracasei GLU70 strain of the present invention, the acid resistance is resistance to pH 2 to 11, the bile resistance is resistance to 0.1 to 1% bile salts, and the heat resistance is resistance to 20 to 60° C.

One example of the present invention relates to a probiotic composition including a Lactobacillus paracasei strain, a culture solution thereof, a concentrate of the culture solution, or a dried product thereof as an active ingredient.

In the present invention, the Lactobacillus paracasei strain may be a Lactobacillus paracasei GLU70 strain.

In the present invention, the Lactobacillus paracasei GLU70 strain may be a Lactobacillus paracasei GLU70 strain with Accession Number KCTC13415BP deposited at the Korean Collection for Type Cultures of the Korea Research Institute of Bioscience and Biotechnology on Dec. 6, 2017.

In the present invention, the culture solution is a culture solution obtained by culturing a strain in a conventional medium, and it may be a culture including cells and/or a cell-free culture solution.

In the present invention, the culture solution may be used as is, or it may be dried or concentrated by a conventional method and used in the form of a concentrate or dried product.

In the present invention, probiotics (live bacteria preparation) are live bacteria, that is, microorganisms that can survive and remain in the gastrointestinal tract when ingested by humans or animals, and refer to microbial preparations that are effective in preventing or treating specific pathological conditions. In general, probiotics are effective in treating and ameliorating various symptoms caused by abnormal fermentation of intestinal flora, and when administered to humans and animals, they concentrate and settle on the walls of the gastrointestinal digestive tract to prevent harmful microorganisms from settling thereon, and produce lactic acid to lower intestinal pH and inhibit the growth of harmful microorganisms.

In the present invention, a probiotic composition may be prepared and administered in various formulations and methods according to the methods known in the art. For example, a probiotic composition of the present invention may be mixed with a carrier commonly used in the pharmaceutical field to be prepared and administered in the form of tablets, troches, capsules, elixirs, syrups, powder, suspensions, or granules.

In the present invention, the carrier may be one or more selected from the group consisting of binders, lubricants, disintegrants, excipients, solubilizers, dispersants, stabilizers, suspending agents, colorants, and fragrances, but is not limited thereto.

In the present invention, the dosage of the probiotic composition may be appropriately selected depending on the absorption of the active ingredient in the body, the inactivation rate, the excretion rate, the age, gender, condition, and severity of the disease of the recipient.

To obtain an effect, the number of bacteria included in the probiotic composition may be 1×10⁸ cells/ml or more, for example, 1×10⁸ cells/ml to 1×10¹³ cells/ml, more specifically 7×10⁹ cells/ml to 7×10¹³ cells/ml, but is not limited thereto.

Another example of the present invention relates to a food composition including a Lactobacillus paracasei GLU70 strain with Accession Number KCTC13415BP, a culture solution thereof, a concentrate of the culture solution, or a dried product thereof as an active ingredient.

In the present invention, the Lactobacillus paracasei GLU70 strain with Accession Number KCTC13415BP, a culture solution thereof, a concentrate of the culture solution, or a dried product thereof may be included in the form of the probiotic composition.

The probiotic composition may be added as is or used together with other foods or food ingredients, and may be appropriately used according to a conventional method.

The mixing amount of the probiotic composition may be suitably determined depending on the purpose of use, for example, prevention, amelioration, or treatment. In general, when manufacturing food or beverages, the probiotic composition of the present invention may be added in an amount of 15 parts by weight or less, preferably 10 parts by weight or less, based on the raw materials, but is not limited thereto. In the case of long-term intake for the purpose of health and hygiene or for the purpose of health control, the mixing amount may be below the above range, and since there is no problem in terms of safety, the mixing amount may be an amount exceeding the above range.

In the present invention, “food” refers to a natural product or processed product containing one or more nutrients, and preferably refers to a product that is processed to be readily eaten. In the conventional sense, the term is used to refer to all of various foods, health functional foods, beverages, food additives, and beverage additives. Examples of the above foods include various foods, beverages, gum, tea, vitamin complexes, functional foods, or the like. In addition, in the present invention, food includes special nutritional foods (e.g., baby formula, infant/toddler food, etc.), health supplement foods, confectioneries (e.g., snacks), processed dairy products, beverages, vitamin complexes, and other health supplement foods, but are not limited thereto. The above foods may be manufactured by conventional manufacturing methods.

In the present invention, the food composition may include sitologically acceptable auxiliary food additives, and may further include appropriate carriers, excipients, and diluents that are commonly used in the manufacture of functional food, but it is not limited thereto.

In the present invention, the food composition may be a functional food composition for improving intestinal microbial flora.

The functional food composition of the present invention may be manufactured and processed in the form of tablets, capsules, powder, granules, liquids, pills, or the like. In the present invention, “health functional food composition” refers to food manufactured and processed using raw materials or ingredients with functionality that is useful to the human body in accordance with Health Functional Food Act No. 6727, and refers to food that is ingested for the purpose of controlling nutrients with respect to the structure and function of the human body or obtaining useful health effects such as physiological effects.

The health functional food of the present invention may include conventional food additives, and suitability as a food additive is determined by the specifications and standards of the item in accordance with the general rules and general test methods of the Food Additives Code approved by the Food and Drug Administration, unless otherwise specified. Items listed in the “Food Additives Code” include, for example, chemical compounds such as ketones, glycine, calcium citrate, nicotinic acid, and cinnamic acid; natural additives such as persimmon color, licorice extract, crystalline cellulose, Kaoliang color, and guar gum; and mixed preparations such as sodium L-glutamate preparations, noodle additive alkaline preparations, preservative preparations, and tar coloring preparations. For example, a health functional food in the form of a tablet may be manufactured by granulating a mixture of an active ingredient of the present invention with excipients, binders, disintegrants, and other additives in a conventional manner, and then adding a lubricant and performing compression molding, or by directly performing compression molding of the mixture. In addition, the health functional food in the form of a tablet may contain flavoring agent, or the like, as needed. Among capsule-type health functional food, hard capsules may be manufactured by filling a conventional hard capsule with a mixture of the active ingredient of the present invention mixed with additives such as excipients, and soft capsules may be manufactured by filling a capsule base such as gelatin with a mixture of the active ingredient of the present invention mixed with additives such as excipients. The soft capsules may contain plasticizers such as glycerin or sorbitol, colorants, preservatives, or the like, as needed. A health functional food in the form of a pill may be manufactured by molding a mixture of the active ingredient of the present invention mixed with excipients, binders, disintegrants, or the like, using a known method, and may be coated with white sugar or other coating agents as needed or the surface may be coated with a material such as starch or talc. A health functional food in the form of granules may be manufactured by granulating a mixture of the active ingredient of the present invention mixed with excipients, binders, disintegrants, or the like, by a known method, and may contain flavoring agents, flavor extracts, or the like, as needed.

Still another example of the present invention relates to an antibacterial composition against pathogenic microorganisms, containing a Lactobacillus paracasei strain, a culture solution thereof, a concentrate of the culture solution, or a dried product thereof as an active ingredient.

In the present invention, the term “antimicrobial” refers to the ability to reduce, prevent, inhibit, or eliminate the growth or survival of bacteria.

In the present invention, the Lactobacillus paracasei strain may be a Lactobacillus paracasei GLU70 strain.

In the present invention, the Lactobacillus paracasei GLU70 strain may be the Lactobacillus paracasei GLU70 strain with Accession Number KCTC13415BP.

In the present invention, the pathogenic microorganism may be one or more selected from the group consisting of Escherichia coli, Staphylococcus aureus subsp. Aureus, Erwinia rhapontici, Staphylococcus epidermidis, Listeria monocytogenes, Shigella flexneri, Shigella sonnei, and Bacillus cereus, but is not limited thereto.

The antibacterial composition according to one embodiment of the present invention may be in the form of food, food additives, feed or feed additives, but is not limited thereto.

The antibacterial composition of the present invention may further include carbohydrates, proteins, lipids, vitamins, and minerals.

In the present invention, carbohydrates, proteins, lipids, vitamins or minerals may be appropriately selected depending on the purpose of use and use.

In the present invention, the carbohydrates may be honey, dextrin, sucrose, palatinose, glucose, fructose, starch syrup, and sugar alcohols such as sorbitol, xylitol, and maltitol, but are not limited thereto.

In the present invention, the proteins may be milk-derived proteins such as casein and whey protein, soy protein, and hydrolyzates of these proteins by animal-derived enzymes such as trypsin and pepsin, as well as neutrase and alkilase, but are not limited thereto.

In the present invention, the lipids may be various vegetable-derived oils such as sunflower oil, rapeseed oil, olive oil, safflower oil, corn oil, soybean oil, palm oil, and coconut oil, including monovalent saturated fatty acids and polyunsaturated fatty acids, medium-chain fatty acids, EPA, DHA, soybean-derived phospholipids, and milk-derived phospholipids, but are not limited thereto.

In the present invention, the minerals may be potassium phosphate, potassium carbonate, potassium chloride, sodium chloride, calcium lactate, calcium gluconate, calcium pantothenate, calcium caseinate, magnesium chloride, ferrous sulfate, and sodium bicarbonate, but are not limited thereto.

Yet another example of the present invention relates to a starter composition for manufacturing fermented food, including a Lactobacillus paracasei GLU70 strain with Accession Number KCTC13415BP, a culture solution thereof, a concentrate of the culture solution, or a dried product thereof as an active ingredient.

In the present invention, the starter for manufacturing fermented food refers to a preparation or composition including microorganisms involved in fermentation for manufacturing fermented food. It is used to provide microorganisms that can grow in fermented food or microorganisms that can grow as a dominant species by adding them when manufacturing fermented food.

When food is manufactured using the starter for manufacturing fermented food, the quality of food may be controlled to be constant by the microorganisms included in the starter for manufacturing fermented food, or a specific purpose, for example, preventing or reducing off-flavors in food may be achieved.

In the present invention, the fermented food may be salted seafood, kimchi, soybean paste, or fermented milk, but is not limited thereto.

In the present invention, the term “starter for manufacturing fermented salted seafood” refers to a preparation or composition including microorganisms involved in fermentation for manufacturing salted seafood. It is used to provide microorganisms that can grow in fermented salted seafood or microorganisms that can grow as a dominant species by adding them when manufacturing salted seafood.

When salted seafood is manufactured using the starter for manufacturing fermented salted seafood of the present invention, by the Lactobacillus paracasei GLU70 strain included in the starter for manufacturing fermented salted seafood, there are an effect of controlling the quality of salted seafood to be constant, an effect of preventing or reducing off-flavors in salted seafood, and an effect of enhancing the savory taste or sweet taste of salted seafood.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a base sequence of a 16S rRNA of a Lactobacillus paracasei GLU70 strain isolated in the present invention;

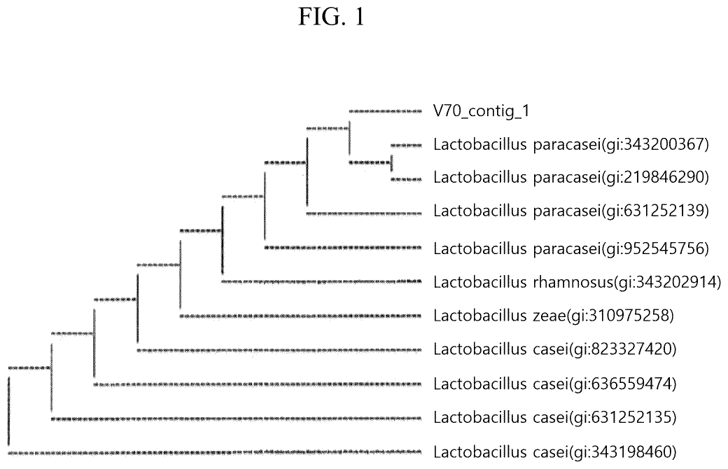

FIG. 2 shows acid resistance analysis results for the Lactobacillus paracasei GLU70 strain of the present invention; and

FIG. 3 shows heat resistance analysis results for the Lactobacillus paracasei GLU70 strain of the present invention.

### DETAILED DESCRIPTION

Hereinafter, the present invention will be described in more detail through the following examples. However, these examples are only for illustrating the present invention, and the scope of the present invention is not limited by these examples.

### Manufacturing Example. Obtaining Lactic Acid Bacteria Through Strain Isolation and Culture Conditions

To isolate lactic acid bacteria having a gluten-degrading ability from various types of salted seafood, various types of salted seafood were finely chopped aseptically, and 1 g thereof was taken, suspended in 9 ml of a sterilized saline solution (0.85% NaCl), and diluted stepwise, and smeared on an MRS solid medium containing 1% gluten. After culturing at 30° C. for 48 hours, only microorganisms that formed a clear zone, which is generated while degrading gluten, around the colonies that appeared were isolated. Next, the isolated microorganisms were smeared on an MRS solid medium and cultured at 30° C. Next, the formed colonies were suspended in a 20% glycerol solution and stored in a −80° C. freezer for use.

### Experimental Example 1. Taxonomic Properties of Lactic Acid Bacteria

The isolated lactic acid bacteria included in a culture solution prepared by culturing in an MRS broth for 24 hours or a single colony of the MRS solid medium (MRS agar) was inoculated into a CHL medium. An API 50 CHL strip consists of 50 tubes containing dried substrate. The test bacteria were inoculated into each capsule and cultured at 37° C. for 48 hours, and then the change in color was checked. After culturing in the capsules of the kit containing 50 carbohydrates as the sole carbon source, it may be determined by the color change whether the test bacteria can utilize the sugar. The purple medium turns yellow as each sugar is utilized, and this was indicated as positive (+). The result values were confirmed in the web software program available on the API website and are shown in Table 1 below.

TABLE 1
API 50 CHL

| | | | |
|---|---|---|---|
| Control | − | Esculin | + |
| Glycerol | − | salicin | + |
| Erythritol | − | D-cellobiose | + |
| D-Arabinose | − | D-maltose | + |
| L-Arabinose | − | D-lactose | + |
| Ribose | + | D-melibiose | − |
| D-xylose | − | D-sacharose | + |
| L-xylose | − | D-trehalose | + |
| Adonitol | − | Inulin | + |
| Methyl-B-D-Xylopyranoside | − | D-melezitose | − |
| D-galactose | + | D-raffinose | − |
| D-glucose | + | Amidon | − |
| D-fructose | + | Glycogene | − |
| D-mannose | + | Xylitol | − |
| L-sorbose | − | Gentiobiose | + |
| L-rhamnose | − | D-turanose | − |
| Dulcitol | − | D-Lyxose | − |
| Inositol | − | D-Tagatose | + |
| D-mannitol | + | D-Fucose | − |
| D-sorbitol | − | L-Fucose | − |
| Methyl-a, D-Mannopyronoside | − | D-Arabitol | − |
| Methyl-a, D-Glucopyranoside | − | L-Arabitol | − |
| N-Acetyl-Glucosamine | + | Gluconate | − |
| Amygdaline | − | 2-Keto-Gluconate | − |
| Arbut in | + | 5-Keto-Gluconate | − |

Identification results: Lactobacillus paracasei (99.8%)
API 50 CHL kit identification.
+: positive;
−: negative

### Experimental Example 2. Genetic Identification of Lactic Acid Bacteria

For genetic identification of the isolated strain, the strain was inoculated into an MRS liquid medium and subjected to secondary subculture for 18 hours at 37° C., and 2 ml of the culture solution was centrifuged at 4,000 rpm for 10 minutes. The cell sediment obtained by centrifugation was washed three times with 0.85% NaCl. After washing, 0.5 ml of lysozyme (10 mg/ml) was added, and the resulting mixture was treated at 37° C. for 1 hour. After adding 20 μl of protease K, a protein decomposition enzyme, and 25 μl of 10% sodium dodecyl sulfate (SDS), the resulting mixture was treated in a water bath at 60° C. for 30 minutes. Afterwards, 1 μl of RNase, an RNA decomposition enzyme, was added, and the resulting mixture was treated at 37° C. for 1 hour. Next, an equivalent amount of phenol-chloroform-isoamyl alcohol was added at a ratio of 25:24:1, and the resulting mixture was suspended and centrifuged at 14,000 rpm for 5 minutes at 4° C. and only the supernatant was collected. This process was repeated twice. 3 M ammonium acetate (pH 4.8) in half the amount of the supernatant and 100% alcohol in twice the amount of the supernatant were added, and the resulting mixture was allowed to stand at −20° C. for 1 hour. A cell sediment was confirmed by centrifugating the mixture at 3,000 rpm for 5 minutes at 4° C. After completely removing the supernatant, 1 ml of 70% ethanol was added, and centrifugation was performed again at 3,000 rpm for 5 minutes at 4° C. After removing the supernatant, the sediment was dried and suspended in a TE buffer or distilled water to isolate DNA. To amplify a 16S rRNA of the microorganisms, the forward primer and the reverse primers shown in Table 2 below were used.

Specifically, 17 μl of distilled water, 1 μl of the forward primer, 1 μl of the reverse primer, and 1 μl of the DNA were added to a PCR premix (Bioneer, Cat No. K-2012) and mixed, and then PCR was performed. The PCR conditions were treatment at 94° C. for 5 minutes, followed by 30 cycles of 1 minute at 94° C., 40 seconds at 62° C., and 40 seconds at 72° C., and the reaction was terminated at 72° C. for 7 minutes. The PCR reaction product was confirmed by electrophoresis using a 0.8% agarose gel. As a result of analyzing the final base sequence and comparing it with the database to identify the lactic acid bacterial strain, the strain isolated by the present invention was found to be Lactobacillus paracasei GLU70, and the strain was deposited at the Korean Collection for Type Cultures of the Korea Research Institute of Bioscience and Biotechnology on Dec. 6, 2017 (Accession Number KCTC13415BP).

TABLE 2

| SEQ ID NO. | Name | Sequence (5′→3′) | Note |
|---|---|---|---|
| 1 | 27f | AGA GTT TGA TCM TGG CTC AG | |
| 2 | 1492r | GGT TAC CTT TGT TAC GAC TT | |

### Experimental Example 3. Acid Resistance Analysis

Lactic acid bacteria, which are widely used as food (and other processed products) or feed additives, affect the balance and metabolism of endogenous microorganisms and play an important role in maintaining intestinal health. In order to exhibit this function as probiotics, lactic acid bacteria must survive without dying under conditions within the digestive tract of humans or animals. Lactic acid bacteria ingested through the mouth must pass through the stomach, in which gastric juice of pH 2 to 3 and various enzymes are present, and go through the duodenum, in which bile is present, to reach the final destination, the intestines, in order that an effect of relieving intestinal disorders may be expected. Among them, to survive at a low pH such as gastric acid in the stomach, lactic acid bacteria must exhibit tolerance to gastric acid (Saarela et al., 2000; Sim et al., 1995).

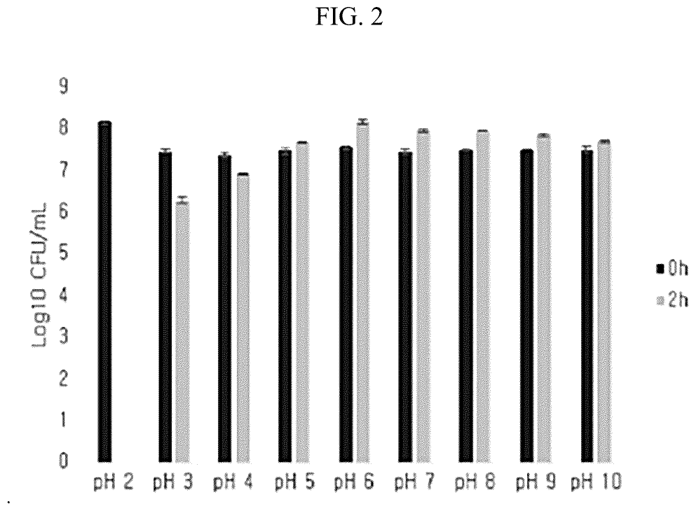

For an acid resistance analysis, 10% of each pre-cultured Lactobacillus paracasei GLU70 strain was inoculated into 10 ml of an MRS medium which was adjusted from pH 2.0 to pH 10.0 in units of pH 1. Next, after allowing the resulting mixture to stand at 37° C. for 2 hours, serial dilution was performed, and 100 μl of the 10⁴, 10⁵, and 10⁶ cultures was smeared on an MRS agar medium. After culturing for 48 hours, the total number of bacterial cells was counted to investigate the resistance to acid, and the results (survival rate by pH) are shown in FIG. 2 and Table 3.

TABLE 3

| pH | Initial cell number | After 2 hour | Survival (%) |
|---|---|---|---|
| 2 | 3.90E+08 | — | 0% |
| 3 | 3.90E+08 | 3.27E+08 | 84% |
| 4 | 3.90E+08 | 3E+08 | 70% |
| 5 | 3.90E+08 | 4E+08 | 97% |
| 6 | 3.90E+08 | 4.13E+08 | 106% |
| 7 | 3.90E+08 | 3.4E+08 | 87% |
| 8 | 3.90E+08 | 2.97E+08 | 76% |
| 9 | 3.90E+08 | 3.03E+08 | 78% |
| 10 | 3.90E+08 | 3.55E+08 | 91% |

survival(%) = final (cfu/ml)/control(cfu/ml) × 100

As can be seen in FIG. 2 and Table 3, the Lactobacillus paracasei GLU70 strain of the present invention showed a high survival rate of 84% after 2 hours at pH 3.0, and was found to die at pH 2.0, but it maintained a high survival rate of 70% or higher under conditions of pH 3.0 or higher. As described in similar research reports, it can be evaluated that the Lactobacillus paracasei GLU70 strain of the present invention has excellent acid resistance and thus has a high possibility of being used in the food industry in the future.

### Experimental Example 4. Heat Resistance Analysis

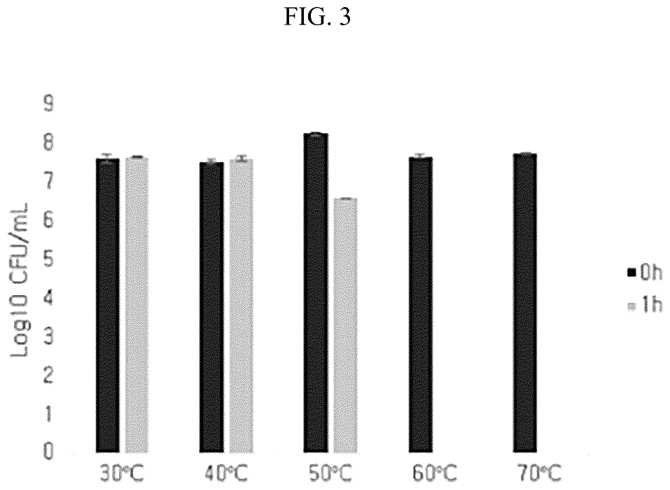

The pre-cultured Lactobacillus paracasei GLU70 strain was inoculated at 10% in 10 ml of an MRS medium. To evaluate resistance in a range from 30° C. to 70° C., the samples were allowed to stand for 1 hour at each temperature and serially diluted, and 100 μl of the 10⁴, 10⁵, and 10⁶ cultures were smeared on an MRS agar medium. After culturing for 48 hours, the total number of the bacterial cells was counted to investigate the resistance to heat. The culture solution cultured in an MRS broth was heat-treated for 2 hours from 30° C. to 70° C. at intervals of 10° C., and then the number of viable cells and heat resistance were confirmed, and the results are shown in FIG. 3 and Table 4.

TABLE 4

| | Initial cell | After | |
|---|---|---|---|
| Temp (° C.) | number | 1 hour | Survival (%) |
| 30 | 1.63E+08 | 1.63E+08 | 100% |
| 40 | 1.63E+08 | 1.63E+08 | 100% |
| 50 | 1.93E+08 | 1.39E+08 | 72% |
| 60 | 1.63E+08 | — | 0% |
| 70 | 1.63E+08 | — | 0% |

As can be seen in FIG. 3 and Table 4, the Lactobacillus paracasei GLU70 strain of the present invention maintained 100% resistance at 30° C. and 40° C. and 72% resistance at 50° C., but showed no resistance above 60° C.

### Experimental Example 5. Analysis of Antibacterial Activity Against Pathogenic Microorganisms

It is known that probiotic lactic acid bacteria inhibit the growth of pathogenic bacteria and spoilage bacteria derived from food under in vitro conditions, and this inhibition mechanism is described as an action of lactic acid produced by lactic acid bacteria, low fatty acids, hydrogen peroxide, diacetyl, bacteriocins, antibiotics, and immunity system stimulation. As a result of Lim et al. (2007) investigating the antibacterial activity of the L. salivarius CPM-7 strain at different incubation times, it was reported that E. coli-inhibiting clear zones were clearly observed from 12 hours, which belongs to the logarithmic phase, and the inhibitory activity continued up to 48 hours.

Therefore, in the present invention, the antibacterial activity of the Lactobacillus paracasei GLU70 strain against eight types of pathogenic microorganisms was investigated. Specifically, 100 μl of eight types of pathogenic microorganisms cultured at 37° C. for 24 hours were smeared on nutrient agar (Difco, USA). Holes with a diameter of 8 mm were drilled in the smeared plate medium, and 200 μl of the culture supernatant was dispensed into each hole. At this time, the culture supernatant was obtained by centrifuging (6000 rpm, 20 minutes, 4° C.) the culture solution of Lactobacillus paracasei cultured for 24 hours in an MRS broth at 37° C. The plate to which the supernatant was added was cultured for 24 hours at the optimal temperature for the growth of each pathogenic microorganism, and then the formation of growth-inhibiting clear zones around the holes was confirmed, and the size of the clear zones was measured. After culturing at 37° C. for 24 hours, the formation of growth-inhibiting clear zones around the disc was confirmed, and the results are shown in Table 5.

TABLE 5

| | 1 | 2 | 3 | 4 | 5 | 6 | 7 | 8 |
|---|---|---|---|---|---|---|---|---|
| strain | E. coli | S. Aureus | E. rhaponitici | S. epidermidis | Listeria | Sh. Flexneri | Sh. sonnei | B. cereus |
| L. paracasei | +++ | +++ | +++++ | ++ | +++ | +++ | ++++ | +++++ |

Degree of clarify of clear zone by growth inhibition: +; 0-2 mm, ++; 2.1-4 mm, +++; 4.1-6 mm, ++++; 6.1-8 mm, +++++; 8.1-10 mm

As can be seen in Table 5, clear zones were formed in all the eight types of pathogenic bacteria. In particular, E. rhapontici and B. cereus produced clear zones of 10 mm, confirming the high antibacterial activity of the Lactobacillus paracasei GLU70 strain, and S. epidermidis showed the smallest inhibition clear zone, and the rest produced clear zones of 5 mm or more, confirming the antibacterial activity of the Lactobacillus paracasei GLU70 strain.

The present invention relates to the use of a Lactobacillus paracasei GLU70 strain, and since the strain of the present invention has probiotic properties and antibacterial activity, it can be used as a probiotic composition, an antibacterial composition, and a starter composition for manufacturing fermented food.

What is claimed is:

1. A method for manufacturing a composition, comprising:
Isolation step for isolating a Lactobacillus paracasei GLU70 strain with Accession Number KCTC13415BP from salted seafood;
Steps for cultivating the Lactobacillus paracasei GLU70 strain with Accession Number KCTC13415BP in a medium; and
Manufacturing step for manufacturing a composition comprising the Lactobacillus paracasei GLU70 strain with Accession Number KCTC13415BP, a culture solution thereof, a concentrate of the culture solution, or a dried product thereof as an active ingredient.

2. The method according to claim 1, wherein the composition is probiotic composition.

3. The method according to claim 1, wherein the composition is a food composition.

4. The method according to claim 3, wherein the food is a health functional food, a food additive, or a processed food.

5. The method according to claim 1, wherein the composition is a starter composition for manufacturing fermented food.

6. The method to claim 5, wherein the fermented food is salted seafood, kimchi, soybean paste, or fermented milk.

7. An antibacterial method against pathogenic microorganisms comprising a composition according to claim 1.

8. The antibacterial method according to claim 7, wherein the pathogenic microorganism is one or more selected from the group consisting of Escherichia coli, Staphylococcus aureus subsp. Aureus, Erwinia rhapontici, Staphylococcus epidermidis, Listeria monocytogenes, Shigella flexneri, Shigella sonnei, and Bacillus cereus.
